(19) United States
(12) Patent Application Publication
(10) Pub. No.: US 2006/0188568 A1
(43) Pub. Date: Aug. 24, 2006

(54) Stable formulations of ace inhibitors and methods for preparation thereof

(75) Inventors: Shailesh Bhamare, PUNE, MAHARASHTRA (IN); Indu Bhushan, Pune (IN); Himadri Sen, Pune (IN)

Correspondence Address: HAMRE, SCHUMANN, MUELLER & LARSON, P.C., P.O. BOX 2902-0902, MINNEAPOLIS, MN, 55402, US

(73) Assignee: LUPIN LIMITED, MUMBAI (IN)

(21) Appl. No.: 10/550,181

(22) PCT Filed: Oct. 30, 2003
(86) PCT No.: PCT/IN03/00346

§ 371 Date: Sep. 21, 2005

(51) Int. Cl. A61K 31/401; A61K 9/20

(52) U.S. Cl. 424/464; 514/423

(57) ABSTRACT

Stabilized pharmaceutical solid composition of ACE inhibitor comprising an ACE inhibitor and a selective dosage formulation thereof comprising of meglumine. The ACE inhibitor selectively combined with a dosage form including essentially the meglumine is surprisingly found to avoid the degradation of ACE inhibitor by such dosage forms especially the commonly used pharmaceutical excepients. In particular, the presence of the meglumine in the dosage form for the active along with the active ACE inhibitor surprisingly avoid the degradation of the ACE inhibitor due to a) cyclization via internal nucleophilic attack to form substituted diketopiperazines, b) hydrolysis of the side chain ester group, and c) oxidation to form products having often unwanted coloration.

### FIELD OF INVENTION

The present invention relates to stable formulations of ACE inhibitors and to a method for their preparation.

### BACKGROUND OF INVENTION

ACE inhibitors, or inhibitors of angiotensin converting enzymes, are drugs useful in the treatment of cardiovascular disorders, especially hypertension.

ACE inhibitors are generally very difficult to formulate into dosage forms, as most ACE inhibitors on contact with some of the commonly used pharmaceutical excipients undergo degradation at accelerated rates due to:
i) cyclization via internal nucleophilic attack to form substituted diketopiperazines,
ii) hydrolysis of the side chain ester group, and
iii) oxidation to form products having often unwanted coloration.

These drugs are therefore not sufficiently stable to enable long shelf life. It is thus generally difficult to select the excipients that enable dosage forms with adequate stability.

Certain stabilized compositions and formulations of ACE inhibitors have been suggested and utilized in the prior art.

U.S. Pat. No. 5,562,921 discloses that stable tablet formulations containing enalapril maleate can be made comprising anhydrous lactose as filler and zinc stearate as lubricant.

U.S. Pat. No. 4,830,853 discloses that ACE inhibitors can be stabilized against oxidation and discoloration by including ascorbic acid or sodium ascorbate in the composition.

U.S. Pat. No. 4,743,450 discloses stable formulations of ACE inhibitors containing alkaline earth metal carbonate and saccharide as stabilizing agents.

WO 03/059388 describes stable formulation of ACE inhibitors comprising only alkaline earth metal carbonate and alkaline earth metal hydrogen phosphate and no saccharide.

U.S. Pat. No. 5,006,344 demonstrates that compositions containing fosinopril sodium are relatively unstable if they comprise magnesium stearate as lubricant, but stability can be improved by use of sodium stearyl fumarate or hygrogenated vegetable oil as lubricant.

Although each of the above patents represent an attempt to overcome the instability problems associated with ACE-inhibitor containing compositions, there still exists a dire need for ACE-inhibitor containing compositions exhibiting improved stability. To this end, the present invention is directed to pharmaceutical compositions of ACE-inhibitors exhibiting improved stability.

### OBJECTS OF THE INVENTION

The object of the present invention is to provide stabilized pharmaceutical compositions comprising ACE-inhibitors which would avoid the instability associated with ACE inhibitors when in dosage forms discussed above.

It is a further object of this invention to disclose stabilized pharmaceutical compositions comprising ramipril and meglumine.

It is another object of the present invention to disclose a process for the preparation of stabilized pharmaceutical compositions comprising an ACE inhibitor.

It is yet another object of the present invention to disclose a stable pharmaceutical composition comprising an ACE inhibitor and selective diluent which would not have problems of compatibility and/or stability usually found in such combination.

### SUMMARY OF THE INVENTION

Thus according to the basic aspect of the present invention there is provided stabilized pharmaceutical solid composition of ACE inhibitor comprising an ACE inhibitor and a selective dosage formulation thereof comprising of meglumine.

Importantly, it is surprisingly found by way of the present invention that if the ACE inhibitor is selectively combined with dosage form including essentially the meglumine, the degradation of ACE inhibitor by such dosage forms especially the commonly used pharmaceutical excepients can be avoided. In other words, the presence of the meglumine in the dosage form for the active along with the active ACE inhibitor surprisingly avoid the degradation of the ACE inhibitor due to
i) cyclization via internal nucleophilic attack to form substituted diketopiperazines,
ii) hydrolysis of the side chain ester group, and
iii) oxidation to form products having often unwanted coloration.

Accordingly, the composition of the invention involving the active ACE inhibitor and the dosage form including essentially the meglumine provide surprising stable and long shelf life for the ACE inhibitor in selective dosage forms.

### DETAILED DESCRIPTION OF THE INVENTION

It is thus possible by way of the above pharmaceutical formulation of present invention to provide an ACE-inhibitor in dosage form including other pharmaceutically acceptable excipients in the presence of meglumine.

The ACE-inhibitor in accordance with present invention may be selected from the group of enalapril, delapril, lisinopril, moxipril, perindopril, ramipril, trandolapril and pharmaceutically acceptable salts thereof. The amount of ACE-inhibitor in the formulation is selected as per its approved dosage strength.

Meglumine is used as a stabilizer. It is an organic base used as pH adjusting agent and solubilizing agent. It is mostly used for parenteral preparations. The ratio of ACE-inhibitor to meglumine is from about 1:0.01 to about 1:2.0 and more preferably from about 1:0.03 to about 1:1.2.

The formulations in accordance with the present invention can due to the selective stability provided by meglumine include other pharmaceutically acceptable excipients selected from amongst diluents and lubricants.

There are many diluents that can be used in pharmaceutical formulations, including for example starch cellulose, calcium sulphate, calcium carbonate, dicalcium phosphate, lactose, dextrose, sucrose, dextrates, mannitol, maltodextrin, methylcellulose, and polyethylene glycol.

However, ACE-inhibitors are incompatible with many of these commonly used pharmaceutical diluents and it is essential to choose a diluent which is compatible with the ACE inhibitors and provide formulations with adequate stability.

According to another aspect of the present invention it has been surprisingly found that better stability of ACE-inhibitors is achieved by using selectively low substituted hydroxypropyl cellulose as a diluent in the dosage formulation.

The ratio of ACE-inhibitor to low substituted hydroxypropyl cellulose used in accordance with the present invention is from about 1:10 to about 1:100.

It was surprisingly found that the combination of meglumine and low substituted hydroxypropyl cellulose or the combination of meglumine with previously known diluents such as pregelatinized starch results in enhanced stability of ACE-inhibitor containing compositions. Incorporation of meglumine along with low substituted hydroxypropyl cellulose or pregelatinized starch produces the stability superior to that of low substituted hydroxypropyl cellulose or pregelatinized starch when used alone.

The lubricant used in accordance with the present invention is selected from amongst stearates such as magnesium stearate, zinc stearate or calcium stearate. Preferably the lubricant is magnesium stearate. It is present in an amount from about 0.2 mg to about 2 mg per tablet or capsule and is more preferably from about 0.5 mg to about 1.5 mg per tablet or capsule.

### EXAMPLES

The objects of the invention and its advantages are explained in greater detail in relation to non-limiting exemplary illustrations of the ACE inhibitor based dosage forms including meglumine of the invention discussed above.

To ascertain the selective stable dosage form for the ACE inhibitors following exemplary preparations with Ramipril as the ACE inhibitor were obtained as per Examples 1 to 5:

| | Example No: | | | | |
|---|---|---|---|---|---|
| | 1 | 2 | 3 | 4 | 5 |
| Ingredients | Amount (mg) | | | | |
| Ramipril | 20 | 20 | 20 | 20 | 20 |
| Pregelatinized Starch | 2000 | 2000 | | | |
| Microcrystalline Cellulose | | | 2000 | | |
| Low substituted Hydroxypropyl | | | | 2000 | 2000 |
| Cellulose | | | | | |
| Meglumine | | 20 | | | 8 |

For each of five examples, the ingredients in the proportions shown were mixed together. The produced mixture was then filled into glass vials and closed with rubber stopper and aluminium seals.

The vials were stored at 60° C. for 15 days and then tested by High Performance liquid Chromatography method (HPLC) to determine assay and degradation products.

The results are summarized in the Table I below:

| | Ramipril Assay | Degradation Products |
|---|---|---|
| Example No. | (%) | (%) |
| 1 | 94.12 | 5.88 |
| 2 | 99.25 | 0.75 |
| 3 | 83.74 | 16.26 |
| 4 | 94.80 | 5.2 |
| 5 | 98.41 | 1.59 |

As can be seen from the Table I above, vials containing meglumine (Ex 2 and 5) exhibited enhanced stability as shown by reduced formation of degradation products (0.75-1.59%) compared with the vials which did not contain meglumine (Ex1, 3 and 4).

The extent of degradation observed in samples containing meglumine either with pregelatinized starch or low substituted hydroxypropyl cellulose was substantially lower than the samples without meglumine.

Surprisingly, low substituted hydroxypropyl cellulose showed higher degree of compatibility unlike observed and reported incompatibility with other celluloses.

It is thus possible by way of the above selective dosage formulation of ACE inhibitors to provide stabilized pharmaceutical compositions comprising ACE-inhibitors which would avoid the instability associated with ACE inhibitors when in dosage forms discussed above. Importantly, the stable pharmaceutical composition comprising an ACE inhibitor and selective diluent would avoid problems of compatibility and/or stability usually found in such combination.

1. A stabilized pharmaceutical solid composition comprising of an ACE inhibitor and meglumine.

2. A stabilized composition of claim 1, where in the ACE inhibitor is selected from the group of enalapril, delapril, lisinopril, moxipril, perindopril, ramipril, trandolapril and pharmaceutically acceptable salts thereof.

3. A stabilized composition of claim 2, wherein the ACE-inhibitor is ramipril.

4. A stabilized composition of claim 3, wherein the amount of ramipril in the composition is from about 1 mg to about 10 mg.

5. A stabilized composition of claim 1, wherein the ratio of ACE-inhibitor to meglumine is from about 1:0.01 to about 1:2.0.

6. A stabilized composition of claim 5, wherein the ratio of ACE-inhibitor to meglumine is preferably from about 1:0.03 to about 1:1.2.

7. A stabilized composition of claim 1, which further comprises of a diluent.

8. A stabilized composition of claim 7, wherein the diluent is selected from amongst low substituted hydroxypropyl cellulose and pregelatinized starch.

9. A stabilized composition of claim 7, wherein the ratio of ACE-inhibitor to diluent is from about 1:10 to about 1:100.

10. A stabilized composition of claim 1 wherein the dosage formulation further comprises of lubricant.

11. A stabilized composition of claim 10, wherein the lubricant is a stearate, which is selected from the group consisting of magnesium stearate, zinc stearate and calcium stearate.

12. A stabilized composition of claim 10, wherein the lubricant is magnesium stearate.

13. A stabilized composition of claim 10, wherein the amount of lubricant in the composition is from about 0.2 mg to about 2 mg.

14. A stabilized composition of claim 10, wherein the amount of lubricant in the composition is from about 0.5 mg to about 1.5 mg.

15. A stabilized pharmaceutical ACE inhibitor composition comprising ramipril and meglumine along with at least one of low substituted hydroxypropyl cellulose, pregelatinized starch and magnesium stearate.

16. A stabilized composition of claim 1 in any dosage form.

17. A stabilized composition of claim 16 wherein the composition is filled into a capsule.

18. A stabilized composition of claim 16 wherein the composition is made into a tablet.

19. A process of preparation of a stable formulation of ACE-inhibitor comprising mixing of the ACE inhibitor with meglumine and optionally at least one of a diluent and a lubricant followed by compressing the mixture to a tablet or filling the mixture into a capsule.

20. The process as claimed in claim 19 wherein the diluent is selected from amongst low substituted hydroxypropyl cellulose and pregelatinized starch.

21. The process as claimed in claim 19 wherein the lubricant, is selected from the group consisting of magnesium stearate, zinc stearate and calcium stearate.

22. The process as claimed in claim 21 wherein the lubricant is magnesium stearate.

23. (canceled)
